(19) United States
(12) Patent Application Publication
(10) Pub. No.: US 2011/0124741 A1
(43) Pub. Date: May 26, 2011

(54) RADIOSENSITIZER COMPOSITIONS COMPRISING SCHISANDRA CHINENSIS(TURCZ.)BAILL AND METHODS FOR USE

(75) Inventors: I Cheng LAI, Taichung City (TW); Charng-Cherng CHYAU, Taichung City (TW); Chien-Cheng CHEN, Taichung County (TW); I-Hsuan LIN, Tainan City (TW); Chien-Chih CHEN, Taipei City (TW); Heng Ju LAI, Taichung City (TW)

(73) Assignee: ETEN BIOTECHNOLOGY LTD., CO., Taichung City (TW)

(21) Appl. No.: 12/950,930

(22) Filed: Nov. 19, 2010

Related U.S. Application Data
(60) Provisional application No. 61/263,011, filed on Nov. 20, 2009.

(51) Int. Cl. A61K 31/09; A61P 35/00

(52) U.S. Cl. 514/719

(57) ABSTRACT

The present invention provides a method of potentiating radiation therapy for treatment of a cancer or tumor comprising administrating to a subject in need thereof a therapeutically effective amount of a radiosensitizer in combination of a radiation therapy to a locus of the cancer or tumor, wherein the radiosensitizer is an extract of Schisandra chinensis (Turcz.) Baill, or the active ingredient isolated therefrom, particularly Schisandrin B.

### FIELD OF THE INVENTION

The present invention is related to a novel radiosensitizer for potentiating radiation therapy for cancers.

### BACKGROUND OF THE INVENTION

Schisandra chinensis (Turcz.) Baill is usually used as Chinese herbal medicine, for example its fruits and seeds. Some compounds were isolated from Schisandra chinensis (Turcz.) Baill, which were regarded to be potentially active components, including, for example, Gomisin O, Epi-gomisin O, Schisandrin, Isoschisandrin, Schizandrol B, Gomisin R, Gomisin J, Gomisin G, Schisantherin A, Gomisin F, Angeloylgomisin P, Tigloylgomisin P, Schisanhenol, Deoxyschisandrin, Gomisin N, Schisandrin B, Gomisin M1, Gomisin M2, Gomisin L1, Gomisin L2, and Schisandrol A, etc. It was reported that Schisandra chinensis (Turcz.) Baill or these compounds were studied on the effectiveness for prevention of neurodegenerative disease and oxidative neural damage, the effect on inhibition of P-glycoprotein, hepatoprotective activities, antioxidant activities, anti-inflammatory and anticancer effect (Ming-Chih Wang et al., J. Sep. Sci. 31:1322-1332, 2008).

Radiation therapy for cancer typically works by attacking rapidly growing cells with highly penetrating ionizing radiation. Unfortunately, radiation therapy does not limit the effects of such treatment to cancer cells, and also affects the surrounding healthy tissue. In addition, tumor cells in a hypoxic environment may be more resistant to radiation damage than those in a normal oxygen environment (Harrison et al., Impact of tumor hypoxia and anemia on radiation therapy outcomes, Oncologist, 7 (6): 492-508, 2002). Thus, radiosensitizers have been developed so as to lower radiation dose to treat the lesion tumor or enhance the effectiveness of radiation therapy.

Some compounds were found to be radiosensitizers that enhance the therapeutic effect when administered during radiation therapy, such as histidine derivatives, halogenated pyrimidine and a hypoxic cell radiosensitizer. However, most of these known radiosensitizers are toxic, which is undesired. Accordingly, it is still desirable to develop new radiosensitizers without toxicity.

### BRIEF SUMMARY OF THE INVENTION

The present invention relates to the discovery of a crude extract obtained from Schisandra chinensis (Turcz.) Baill and the compounds contained that can make cancer or tumor cells more sensitive to radiation therapy.

In one aspect, the present invention provides a method of potentiating radiation therapy for treatment of a cancer or tumor comprising administrating to a subject in need thereof a therapeutically effective amount of a radiosensitizer in combination of a radiation therapy to a locus of the cancer or tumor, wherein the radiosensitizer is an extract of Schisandra chinensis (Turcz.) Baill.

In another aspect, the present invention further provides a method of potentiating radiation therapy for treatment of a cancer or tumor comprising administrating to a subject in need thereof a therapeutically effective amount of a radiosensitizer in combination of a radiation therapy to a locus of the cancer or tumor, wherein the radiosensitizer is a compound of formula (I):

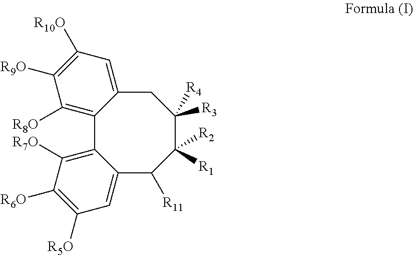

wherein one of R₁ to R₁₀ is H or C₁-C₃ alkyl, and R₁₁ is —OH, —O-benzoyl, —O-angeloyl, or —O-tigloyl, wherein R₅ and R₆ or R₉ and R₁₀ may be taken together with the adjacent oxygen and the carbon to which the oxygen atoms are bound to form 1,3, dioxole.

In one embodiment of the invention, the radiosensitizer is the active ingredients contained in Schisandra chinensis (Turcz.) Baill, particularly Schisandrin B.

Further objects and advantages of the invention will become apparent for the following description.

### BRIEF DESCRIPTION OF THE DRAWINGS

The foregoing summary, as well as the following detailed description of the invention, will be better understood when read in conjunction with the appended drawings. It should be understood, however, that the invention is not limited to the precise arrangements and instrumentalities shown.

In the drawings:

FIG. 1 provides a diagram showing the cell viability (%) of HepG2 treated with various concentrations of ES800 (0, 12.5, 25, 50, 100, and 200 μg/ml) for 72 hours;

FIG. 2 provides a diagram showing the percentage of Annexin V⁺/PI⁺ HepG2 cells treated with 0.008% DMSO (“control”), 8 Gy alone (“8 Gy”), and 8 Gy combined with 40 μg/ml ES800 (“40 μg/ml+RT”), or 80 μg/ml ES800 (“80 μg/ml+RT”);

FIG. 3A-D provides the diagrams showing the expression level of Bcl-2 (A), p21 (B), caspase 9 (cleaved form)(C), and β-actin (D) in Hep2G cells: a: control; b: treated with 40 μg/ml ES800; c: treated with 80 μg/ml ES800; d: exposed at 8 Gy of radiation alone; e: exposed at 8 Gy of radiation combined with 40 μg/ml ES800; and f: exposed at 8 Gy of radiation combined with 80 μg/ml ES800; wherein * represents p<0.05 compared with the control group; ** represents p<0.05 compared to 8 Gy group;

FIG. 4 provides a diagram showing survival fraction of HepG2 treated with 25 μg/ml ES800 exposed at 0, 2, 4 and 6 Gy of radiation respectively in a colony formation assay; wherein * represents p<0.05 compared with the control group;

FIG. 5 provides a diagram of a colony formation assay showing the survival fraction of HepG2 treated with 25 μg/ml ES800, 160 μM CPT-11, or 320 μM CPT-11 in combination of irradiation at 0, 2, and 4 Gy respectively;

FIG. 6 provides a diagram of a colony formation assay showing the survival fraction of U87 MG treated with 25 μg/ml or 50 μg/ml ES800 in combination of irradiation at 0, 2, 4 and 6 Gy; wherein * represents p<0.05 compared with the control group;

FIG. 7 provides a diagram showing the percentage of Annexin V⁺/PI⁺ HepG2 cells treated with 0.008% DMSO (“control”), 12 μg/ml Schisandrin B (“ShiB 12 μg/ml”), 24 μg/ml Schisandrin B (“ShiB 24 μg/ml”), exposed at 8 Gy alone (“8 Gy”), and exposed at 8 Gy in combination with 40 μg/ml ES800 (“RT+ES800 40 μg/ml”), 80 μg/ml ES800 (“RT+ES800 80 μg/ml”), 12 μg/ml Schisandrin B (“RT+ShiB 12 μg/ml”), or 24 μg/ml Schisandrin B (“RT+ShiB 24 μg/ml”); wherein # represents p<0.05 compared with the control group; ## represents p<0.05 compared to 12 μg/ml Schisandrin B alone; ### represents p<0.05 compared to 24 μg/ml Schisandrin B alone; and * represents p<0.05 compared with 8 Gy group; and

FIG. 8A and FIG. 8B provide the diagrams showing the expression level of caspase 3 (cleaved form)(A), and β-actin (B) in Hep2G cells, respectively (a: control; b: treated with 12 μg/ml Schisandrin B; c: treated with 24 μg/ml Schisandrin B; d: exposed at 8 Gy alone; e: exposed at 8 Gy in combination with 12 μg/ml Schisandrin B; and f: exposed at 8 Gy in combination with 24 μg/ml Schisandrin B); wherein * represents p<0.05 compared with the control group; ** represents p<0.05 compared to 8 Gy group.

### DETAILED DESCRIPTION OF THE INVENTION

Unexpectedly, it is found in the present invention that Schisandra chinensis (Turcz.) Baill and its active ingredients, particularly Schisandrin B, can make cancer or tumor cancers more sensitive to radiation therapy.

Accordingly, the present invention provides a method of potentiating radiation therapy for treatment of a cancer or tumor comprising administering to a subject in need thereof a therapeutically effective amount of a radiosensitizer in combination of a radiation therapy to a locus of the cancer or tumor, wherein the radiosensitizer is an extract of Schisandra chinensis (Turcz.) Baill.

In one embodiment of the invention, the cancer cells, such as HepG2 that is a human liver carcinoma cell line, were treated with the extract of Schisandra chinensis (Turcz.) Baill (ES800) in combination with a radiation therapy (8 Gy), as compared with the radiation therapy alone, and the results showed that the extract of Schisandra chinensis (Turcz.) Baill (ES800) enhanced the effect on the death of cancer cells caused by a radiation therapy.

According to the invention, the extract of Schisandra chinensis (Turcz.) Baill can be prepared by a process comprising the steps of: (a) extracting Schisandra chinensis (Turcz.) Baill with water to obtain a water insoluble fraction; (b) extracting the water insoluble fraction obtained in step (a) with alcohol-based solvent to obtain an alcohol extract; and (c) removing the alcohol-based solvent from the alcohol extract obtained in step (b).

In one embodiment of the present invention, Schisandra chinensis (Turcz.) Baill was dried, ground, and boiled in water for a period of time, such as 1 hour; then, the residues was collected to obtain a water insoluble fraction of Schisandra chinensis (Turcz.) Baill. The water insoluble fraction was further extracted by alcohol, such as ethanol, to obtain an alcohol extract. Optionally, the water insoluble fraction can further be dried by any conventional methods, i.e. lyophilization or heating by a drier, before extracting with alcohol. In Example 1, the ethanol was further removed from the alcohol extract by such as lyophilization, and the extract of Schisandra chinensis (Turcz.) Baill was designated as ES800.

As used herein, the term “radiosensitizer” refers to an agent that make cancer or tumor cells more sensitive to radiation therapy than radiation therapy alone. Accordingly, the same anti-tumor effect can be achieved at a lower radiation dose by co-administration of a radiosensitizer during radiation therapy. The term “therapeutically effective amount” refers to the amount of attaining the above effect as desired. The actual amount to be administrated can vary in accordance with the age, size, and condition of a subject to be treated, depending at the discretion of medical professions.

According to the invention, the extract of Schisandra chinensis (Turcz.) Baill may be constituted into any form suitable for the mode of administration as selected. For example, compositions suitable for oral administration include solid forms, such as pills, capsules, granules, tablets, and powders, and liquid forms, such as solutions, syrups, elixirs, and suspensions. The emulsion composition may be administered by injection or infusion into a vein (intravenous, IV), a muscle (intramuscular, IM), or under the skin (subcutaneous, SC). Preferably, the extract of Schisandra chinensis (Turcz.) Baill is administered orally.

The term “radiation therapy” as used herein refer to is a medical use of ionizing radiation for treatment of cancers or tumor, particularly maligant cells. Normally, the radiation therapy comprises a direct radiation or irradiation, for example X-ray radiation, to the locus of cancer or tumor. However, there is no way to avoid sparing normal tissues (such as skin or organs which the radiation must pass through) or the health tissues surrounding the locus of cancer or tumor to be exposed for radiation or irradiation. Therefore, a lower dose of radiation is desired to reduce the damages to normal and/or health tissues. A co-administration of a radiosensitizer that makes cancer or tumor cells more sensitive to radiation therapy is one approach to lower the radiation dose or enhance the effectiveness of the radiation therapy.

In the invention, the radiation therapy and administration of the radiosensitizer may be performed simultaneously during the course or period of treatment, or the radiosensitizer may be administrated prior to or after the radiation therapy. The radiation conditions may be appropriately selected by a medical practitioner or other professionals, depending on a type of a radiation source, radiation method, radiation site and radiation period, the health state and disease history of a subject to be irradiated, as being well-known in the filed of radiation therapy. The radiation conditions include type, dose and numbers of dose fractions, which may be determined according to the standard procedures, or conventional radiation therapies.

According to the invention, the extract of Schisandra chinensis (Turcz.) Baill, such as ES800, can be used as a radiosensitizer for any kind of cancers or tumors, particularly solid cancers, such as liver cancer or brain cancer.

It is also found in the present invention that the active ingredients isolated from Schisandra chinensis (Turcz.) Baill are effective in making cancer or tumor cells more sensitive to radiation therapy. Accordingly, the present invention in another aspect provides a method of potentiating radiation therapy for treatment of a cancer or tumor comprising administering to a subject in need thereof a therapeutically effective amount of a radiosensitizer in combination of a radiation therapy to a locus of the cancer or tumor, wherein the radiosensitizer is a compound of formula (I).

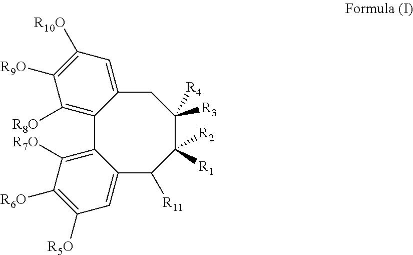

wherein one of R₁ to R₁₀ is H or C₁-C₃ alkyl, and R₁₁ is —OH, —O-benzoyl, —O-angeloyl, or —O-tigloyl, wherein R₅ and R₆ or R₉ and R₁₀ may be taken together with the adjacent oxygen and the carbon to which the oxygen atoms are bound to form 1,3, dioxole.

Examples of the compounds of Formula (I) may include but be not limited to Gomisin O, Epi-gomisin O, Schisandrin, Isoschisandrin, Schizandrol B, Gomisin R, Gomisin J, Gomisin G, Schisantherin A, Gomisin F, Angeloylgomisin P, Tigloylgomisin P, Schisanhenol, Deoxyschisandrin, Gomisin N, Schisandrin B, Gomisin M1, Gomisin M2, Gomisin L1, Gomisin L2, and Schisandrol A. The above mentioned compounds are known, and can be prepared, for example, according to the method described in Ming-Chih Wang et al., J. Sep. Sci. 2008, 31:1322-1332. Based on Formula (I), the values of R₁-R₁₁ for these compounds are given in Table 1:

TABLE I

| Chemical Name | MW | R1 | R2 | R3 | R4 | R5 | R6 | R7 | R8 | R9 | R10 | R11 |
|---|---|---|---|---|---|---|---|---|---|---|---|---|
| Gomisin O | 416 | CH₃ | H | CH₃ | H | CH₃ | CH₃ | CH₃ | CH₃ | —CH₂— | | —OH |
| Epi-gomisin O | 416 | CH₃ | H | CH₃ | H | CH₃ | CH₃ | CH₃ | CH₃ | —CH₂— | | —OH |
| Schisandrin | 432 | OH | CH₃ | H | CH₃ | CH₃ | CH₃ | CH₃ | CH₃ | CH₃ | CH₃ | |
| Isoschisandrin | 432 | H | CH₃ | OH | CH₃ | CH₃ | CH₃ | CH₃ | CH₃ | CH₃ | CH₃ | |
| Schizandrol B | 416 | OH | CH₃ | H | CH₃ | CH₃ | CH₃ | CH₃ | CH₃ | —CH₂— | | |
| Gomisin R | 400 | CH₃ | H | CH₃ | H | —CH₂— | | CH₃ | CH₃ | —CH₂— | | —OH |
| Gomisin J | 388 | H | CH₃ | H | CH₃ | H | CH₃ | CH₃ | CH₃ | CH₃ | H | |
| Gomisin G | 536 | OH | CH₃ | CH₃ | H | —CH₂— | | CH₃ | CH₃ | CH₃ | CH₃ | —O-Benzoyl |
| Schisantherin A | 536 | OH | CH₃ | CH₃ | H | CH₃ | CH₃ | CH₃ | CH₃ | —CH₂— | | —O-Benzoyl |
| Gomisin F | 514 | OH | CH₃ | CH₃ | H | —CH₂— | | CH₃ | CH₃ | CH₃ | CH₃ | —O-Angeloyl |
| Angeloylgomisin P | 514 | CH₃ | OH | CH₃ | H | CH₃ | CH₃ | CH₃ | CH₃ | —CH₂— | | —O-Angeloyl |
| Tigloylgomisin P | 514 | CH₃ | OH | CH₃ | H | CH₃ | CH₃ | CH₃ | CH₃ | —CH₂— | | —O-Tigloyl |
| Schisanhenol | 402 | H | CH₃ | H | CH₃ | CH₃ | CH₃ | CH₃ | H | CH₃ | CH₃ | |
| Deoxyschisandrin | 416 | H | CH₃ | H | CH₃ | CH₃ | CH₃ | CH₃ | CH₃ | CH₃ | CH₃ | |

| | | | | | | | | | | |
|---|---|---|---|---|---|---|---|---|---|---|
| Gomisin N | 400 | H | CH₃ | H | CH₃ | —CH₂— | CH₃ | CH₃ | CH₃ | CH₃ |
| Schisandrin B | 400 | H | CH₃ | H | CH₃ | —CH₂— | CH₃ | CH₃ | CH₃ | CH₃ |
| Gomisin M1 | 386 | H | CH₃ | H | CH₃ | —CH₂— | CH₃ | H | CH₃ | CH₃ |
| Gomisin M2 | 386 | H | CH₃ | H | CH₃ | —CH₂— | H | CH₃ | CH₃ | CH₃ |
| Gomisin L1 | 386 | H | CH₃ | H | CH₃ | —CH₂— | CH₃ | H | CH₃ | CH₃ |
| Gomisin L2 | 386 | H | CH₃ | H | CH₃ | —CH₂— | CH₃ | CH₃ | CH₃ | H |

| | | | | | | | | | | | |
|---|---|---|---|---|---|---|---|---|---|---|---|
| Schisandrol A | 433 | OH | CH₃ | H | CH₃ | CH₃ | CH₃ | CH₃ | CH₃ | —CH₂— | —OH |

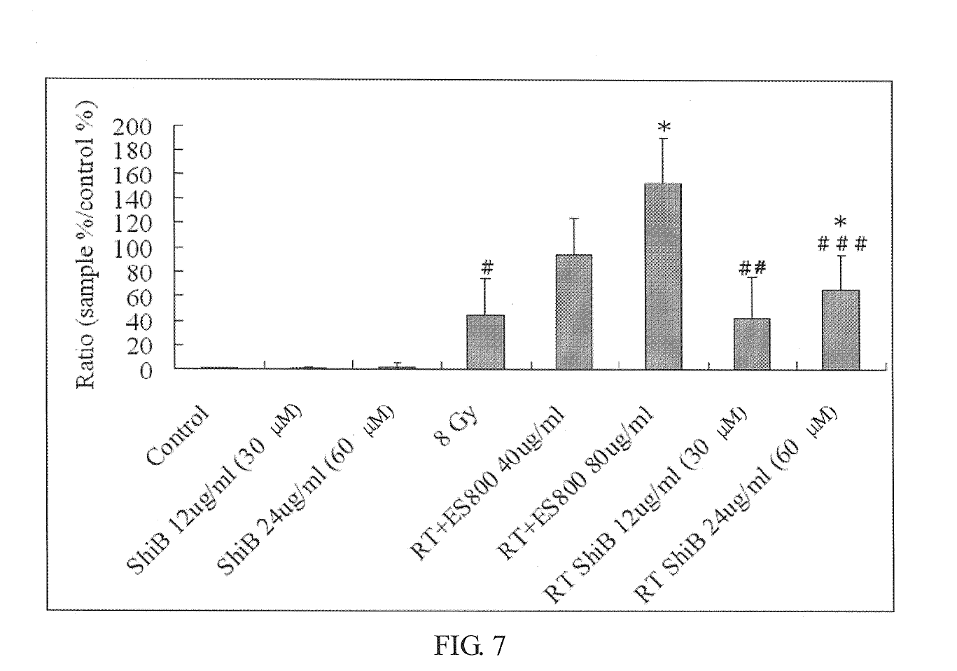

In one example of the present invention, the active ingredient is Schisandrin B. It was evidenced in the example that Schisandrin B in combination of radiation therapy provides an enhanced effect in inhibition of the growth of cancer cells, as compared with the radiation therapy alone (see FIG. 7).

The present invention is further illustrated by the following examples, which are provided for the purpose of demonstration rather than limitation.

### Example 1

### Preparation of the Extract of Schisandra Chinensis (Turcz.) Baill

Dried sample of Schisandra chinensis (Turcz.) Baill (100 g) brought from Sun Ten Pharmaceutical Corporation (Taipei, Taiwan, R.O.C.) was grinded and added into 2000 mL double distilled water (ddH₂O). The immersed sample was then boiled and stirred at 400 rpm for 1 hr of reflux extraction. The step above was repeated three times. The combined solution was then performed a vacuum filtration and the residues was collected to obtain a water insoluble fraction. The water insoluble fraction was then lyophilized and extracted with 95% ethanol (1:10 (v/v)). After 10 min of sonication at the room temperature, the mixture was filtered to collect the alcohol extract. The alcohol extract was then evaporated to dryness, such as by lyophilization. The final product was designated as ES800 and stored for the following experiments.

Schisandrin B was prepared according to the procedure reported by Ming-Chih Wang et al., J. Sep. Sci. 2008, 31: 1322-1332.

### Example 2

### In Vitro Study of ES800 on HepG2

Culturing of HepG2

HepG2 was purchased from Food Industrial Research and Development Institute (Taiwan, R.O.C.) and was cultured with Dulbecco's modified eagle's medium (DMEM, HyClone, Logan, Utah, U.S.A) containing 10% fetal bovine serum (FBS) (Biological industries, Ashrat, Israel) and 10,000 U/ml penicillin-streptomycin (HyClone) under 5% CO2, statured humidity, at 37° C.

Evaluation of the Effect of ES800 on the Survival Rate of HepG2

The aim of this experiment was to evaluate the maximal inhibitory concentration (IC) of HepG2 against ES800, or ES800 in combination of a radiation. HepG2 cells were seeded in 96-well microplate (4,000 cells/well) for 24 hours. Various concentrations of ES800, i.e. 12.5, 25, 50, 100 and 200 μg/ml, were added into the culture medium, wherein 0.008% DMSO was added to the control group. After a 72-hour incubation, the cells survival rates were determined by MTT assay and calculated by a formula below:

Cell survival rate=[(Average of absorption on experimental group)÷(Average of absorption on control group)]×100%

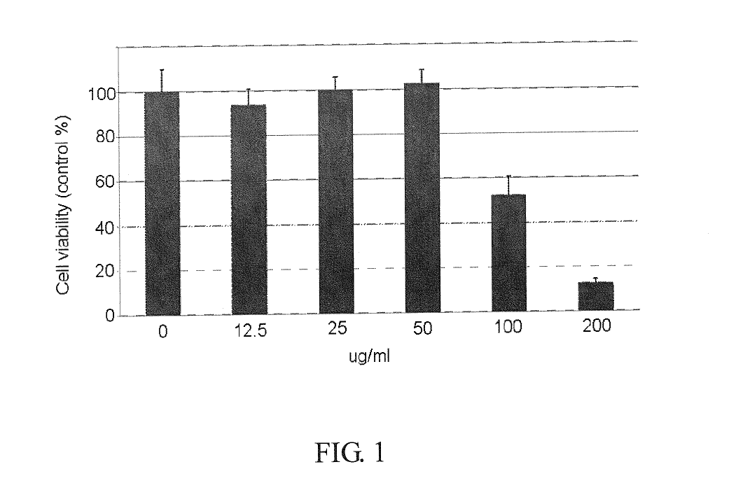

As shown in FIG. 1, 50 μg/ml ES800 was nontoxic to HepG2. Therefore, ES800 at the concentration of 40 μg/ml (IC 12.5) or 80 μg/ml (IC 25) EC800 was used for the in vitro studies.

### Example 3

### In Vitro Study of ES800 as a Radiosensitizer

HepG2 cells were seeded in 6-cm dish (2.5×10⁵ cells/dish) for a 24-hour incubation. Various concentration of ES800 (40 μg/ml or 80 μg/ml) were added into the culture medium, and then incubated for another 24 hours, wherein 0.008% DMSO was added to the control group. The cells were exposed at 8Gy of radiation (Linear accelerator, Philips SL-18), and incubated for another 48 hours. Subsequently, HepG2 were collected and washed by 5 ml Dulbecco's phosphate buffered saline (D-PBS), and then fixed with 70% ethanol at 4° C. overnight. The fixed cells were washed by 5 ml D-PBS, and added 0.5 ml propidium iodide solution (50 μg/ml of propidium iodide (Sigma), 50 μg/ml of RNase A, and 0.1% Triton X-100 in D-PBS) for 30 min of dyeing away from light. The analysis was conducted by Epics XL flow cytometry (Beckman Coulter, Taipei, Taiwan) and shown in Table II.

TABLE II

| | Cell Cycle | | |
|---|---|---|---|
| Group | G0/G1 (%) | S (%) | G2/M (%) |
| Control | 52.23 ± 3.10 | 23.07 ± 1.96 | 24.70 ± 1.15 |
| 8 Gy | 63.13 ± 0.75^{#} | 10.12 ± 0.60^{##} | 26.83 ± 0.59 |
| ES800 40 g/ml + 8 Gy | 66.40 ± 2.62 | 11.87 ± 1.24 | 21.73 ± 3.79 |
| ES800 80 g/ml + 8 Gy | 68.50 ± 0.92* | 6.79 ± 1.31 | 24.70 ± 1.25* |

^{#}represented p < 0.05 compared with the control group;
^{##}represented p < 0.01 compared with the control group;
*represented p < 0.05 compared with the 8 Gy group.

As shown in Table II, the death of the cancer cells exposed at 8 Gy in combination with a treatment with 80 μg/ml ES800 was significantly increased in terms of the percentages of G0/G1, as compared with the treatment with a radiation alone, wherein G0 means the cell cycle at G0 phase; G1 means the cell cycle at G1 phase; S means the cell cycle at synthesis phase; G2 means the cell cycle at G2 phase; and M means the cell cycle at mitotic phase. It is well known that G0/G1 arrest might lead to DNA repair or induce cancer cells apoptosis. Therefore, ES800 evidently showed potentiality on cancer therapy being as a radiosensitizer.

### Example 4

### In Vitro Study of ES800 on its Ability of Inducing Apoptosis

Detection of Annexin V⁺ and PI^{+/-} Cells

Annexin V is a 35-36 kDa Ca2+ dependent phospholipid-binding protein that has a high affinity for the membrane phospholipid phosphatidylserine (PS), and binds to cells with exposed PS. Since externalization of PS occurs in the earlier stages of apoptosis, Annexin V staining can identify apoptosis at an earlier stage than assays based on nuclear changes such as DNA fragmentation. A vital dye such as propidium iodide (PI) is typically used in conjunction with Annexin V to identify the viability of the cells. For example, cells that are considered viable are Annexin V and PI negative; cells that are in early apoptosis are Annexin V positive and PI negative; and cells that are in late apoptosis or already dead are both Annexin V and PI positive, wherein the membranes of dead and damaged cells are permeable to PI. The Annexin V staining assay was performed by Annexin V-FITC-Kit purchased from Beckman Coulter, Inc (U.S.A). The protocol was conducted according to the manufacturing manuscript.

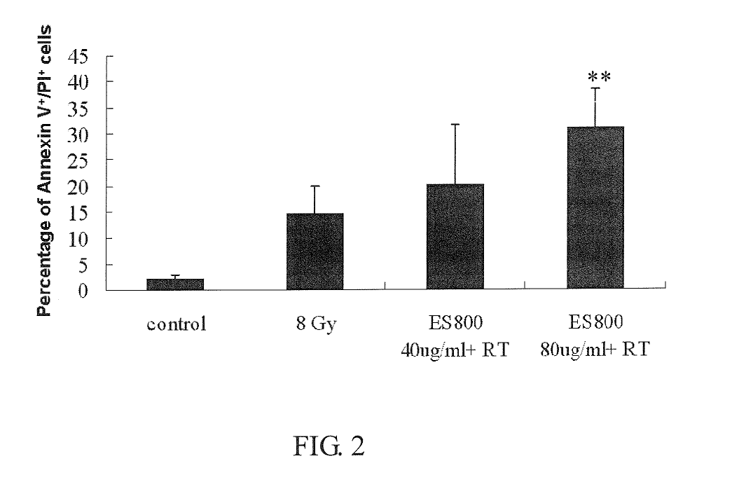

HepG2 cells were seeded in 6-cm dish (2.5×10⁵ cells/dish) for 24 hours of incubation. Various concentration of ES800 (40 or 80 μg/ml) were added into the culture medium, and then incubated for another 24 hours, wherein 0.008% DMSO was added to the control group. The cells were exposed at 8 Gy of radiation (Linear accelerator, Philips SL-18), and incubated for another 48 hours. After collection, the cells were washed by pre-cold PBS and centrifuged at 500×g. The supernatant was removed and the cells were re-suspended with binding buffer. 1 μl Annexin V-FITC solution and 5 μl PI solution were added into the cell suspension and reacted away from light on ice for 15 min. Finally, 400 μl pre-cold binding buffer was added and the samples were analyzed by flow cytometer within 30 min. The percentages of Annexin V⁺ and PI^{+/-} double staining cells were plotted in FIG. 2.

As shown in FIG. 2, the treatment of ES800 in combination with 8 Gy of irradiation provided better effect on inducing apoptosis of cancer cells than the radiation alone.

Results of Western Blot

HepG2 cells were seeded in 6-cm dish (2.5×10⁵ cells/dish) for 24 hours of incubation. Various concentration of ES800 (40 or 80 μg/ml) were added into the culture medium, and then incubated for another 24 hours, wherein control group was added 0.008% DMSO. The cells were exposed at 8Gy of radiation (Linear accelerator, Philips SL-18), and incubated for another 48 hours. After collection, the cells were washed by PBS for three times, and then centrifuged at 360×g for 5 min. After removing the supernatant, 120 μl CelLytic-M (Sigma) and 1 μl Protease Inhibitor Cocktail (Sigma) were added into the pellet to suspend the cells and then the suspension was incubated at 4° C. for 30 min. The total protein was collected from the supernatant by centrifugation at 27210×g for 10 min.

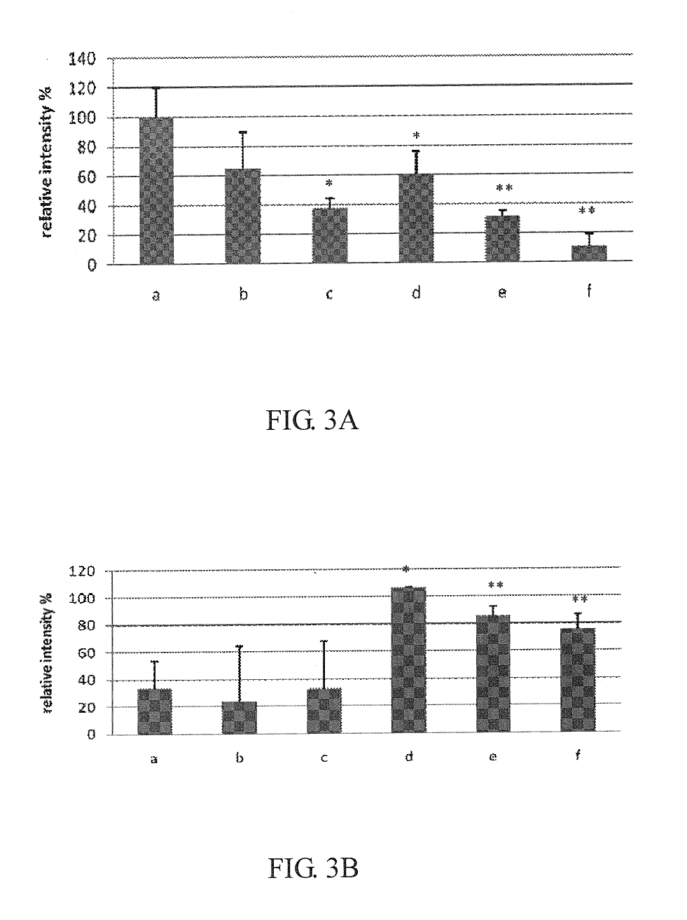

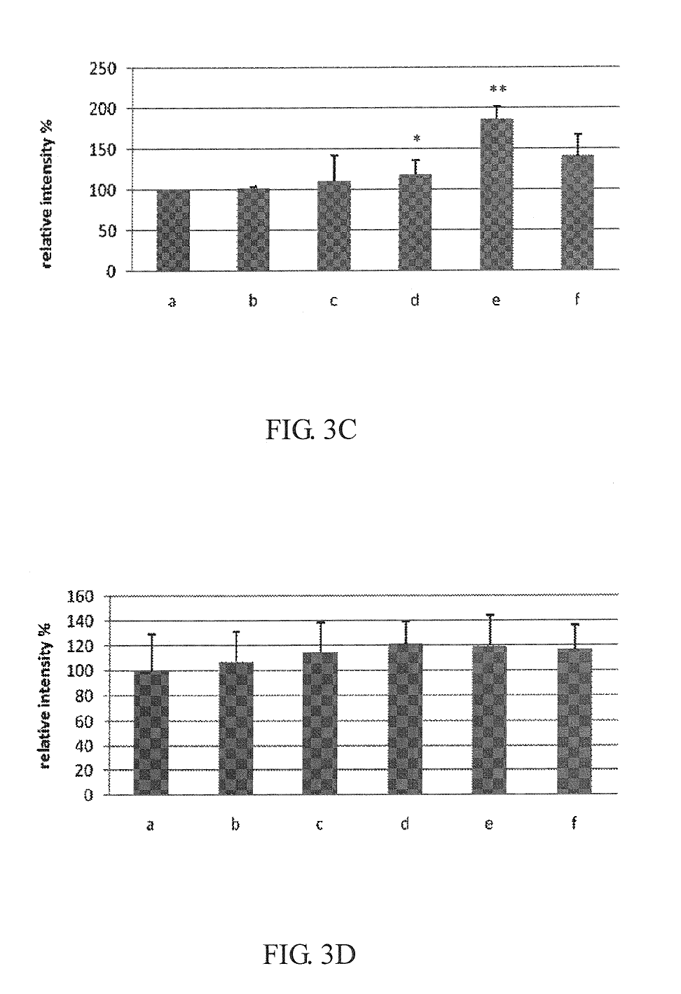

20-30 g protein was loaded to run an SDS-PAGE. The gel was then transferred to PVDF and immersed into TTBS solution (2.42 g Tris base/8 g NaCl/0.1% Tween-20/per liter) containing 5% silk milk for blocking. The membrane was reacted with each primary antibody against p21, Bcl-2, caspase 9 (cleaved form), caspase 3 (cleaved form) and β-actin overnight at 4° C. After the reaction, the membrane was washed by TTBS solution three times, and then reacted with appropriate secondary antibody for another 30-min incubation. The final product was placed into chemiluminescence reagents (Perkin Elmer Life Science) for 1 min, and exposed under X-ray to a film. The bands were quantified by GE ImageMaster 2D Platinum Software. The results were shown in FIGS. 3A-3D, wherein β-actin served as an internal control.

In the study, Bcl-2 and p21 served inhibitors of apoptosis. As shown in FIGS. 3A and 3B, these proteins produced by HepG2 were significantly decreased after a treatment with ES800 in combination of radiation, as compared to the radiation alone. On the other hand, the expression level of caspase 9 in HepG2 when treated with ES800 in combination of a radiation significantly increased as compared to that treated with the radiation alone (FIG. 3C). Given the above, it was evidenced that ES800 provided an effect as a radiosensitizer to make the cancer cells more sensitive to radiation.

### Example 5

### In Vitro Study of ES800 by a Clonogenic Assay

2.6×10⁵ cells of HepG2 were seeded in 6-cm dish for 24 hour-incubation and the medium was replaced with fresh medium containing 25 μg/ml ES800 respectively for another 2-hour incubation. Control group was left untreated. The cells were then exposed to a radiation at 0, 2, 4, and 6 Gy, and 200, 400, 800 and 1600 cells were reseeded in 6-cm dish with fresh medium. After 14-day incubation, the cells were stained with 5% giemsa solution and the cells numbers were counted.

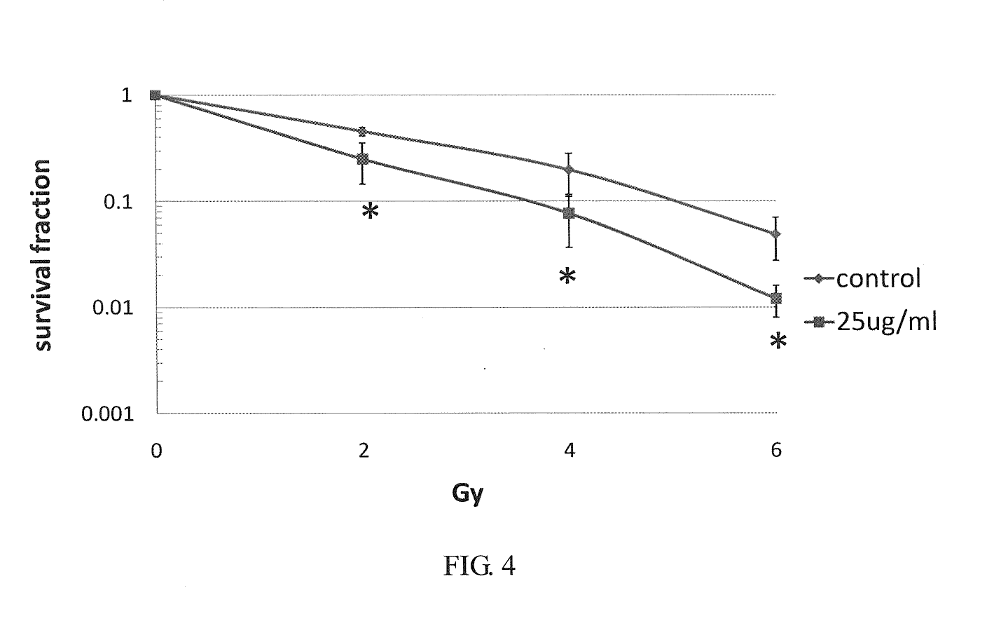

As shown in FIG. 4, the survival fractions of HepG2 exposed at 2, 4 or 6 Gy in combination with the treatment of 25 μg/ml ES800 was significantly lower that of the control group (0 μg/ml ES800).

CPT-11 (Irinotecan) is a drug used for treatment of cancer. It is a semi-synthetic analogue of the natural alkaloid camptothecin, which prevents DNA from unwinding. CPT-11 is often used in colon cancer, particularly in combination with other chemotherapy agents. In the following experiment, a clonogenic assay was also conducted to demonstrate the effects of ES800 and CPT-11 on treatment of cancer.

2.6×10⁵ cells of HepG2 were seeded in 6-cm dish for 24 hours of incubation and the medium was replaced with fresh medium containing 25 μg/ml ES800, 160 μM or 320 μM CPT-11 respectively for another 2 hours of incubation. Control group was left untreated. The cells were then exposed to radiation at 0, 2, and 4 Gy, and 200, 400, 800 and 1600 cells were reseeded respectively in 6-cm dish with fresh medium. After 14 days of incubation, the cells were stained with 5% giemsa solution and the cells numbers were counted.

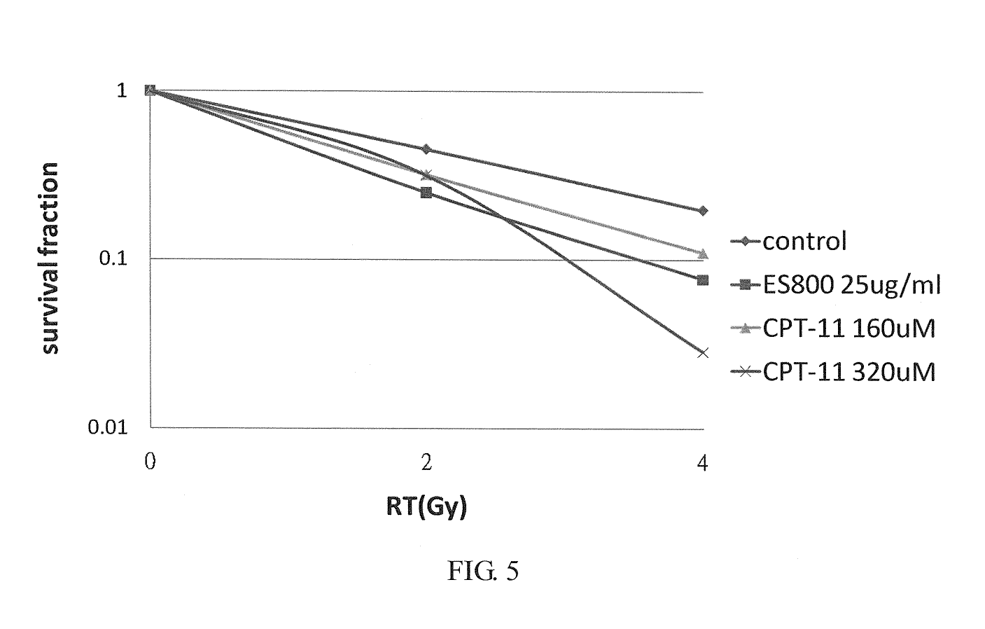

As shown in FIG. 5, ES800 and CPT-11 provided similar effects on decreasing survival fraction of HepG2 at 2 Gy. In combination of a radiation of 4 Gy, ES800 at 25 μg/ml exhibited better activity in killing cancer cells than 160 μM CPT-11.

Clinical cancer drugs are known to be expensive and have serious side effects. For example, the adverse effects of CPT-11 are severe diarrhea and extreme suppression of the immune system. However, in the radiation therapy in combination of the treatment with ES800, the dose of ES800 will be significantly lower than that commonly used in conventional cancer drugs, such as CPT-11, to provide the same effectiveness for cancer treatment but without side effect, and ES800 is cheaper.

### Example 6

### In Vitro Study of ES800 on U87 MG

Culturing of U87 MG

U87 MG was purchased from Food Industrial Research and Development Institute (Taiwan, R.O.C.) and was cultured with Minimum essential medium (MEM, HyClone, Logan, Utah, U.S.A) containing 10% fetal bovine serum (FBS) (Biological industries, Ashrat, Israel), 10,000 U/ml penicillin-streptomycin (HyClone), 1.5 g/L sodium bicarbonate, 0.1 mM non-essential amino acids, and 0.1 mM sodium pyruvate under 5% CO2, statured humidity, at 37° C.

Evaluation of the Effect of ES800 on U87 MG Determined by a Clonogenic Assay

2.6×10⁵ cells of U87 MG were seeded in 6-cm dish for 24 hours of incubation and the medium was replaced with fresh medium containing 25, or 50 μg/ml ES800 for another 2 hours of incubation. Control group was left untreated. The cells were then exposed to radiation of 0, 2, 4, and 6 Gy, and 200, 400, 800 and 1600 cells were reseeded respectively in 6-cm dish with fresh medium. After 14-day incubation, the cells were stained with 5% giemsa solution and the cells numbers were counted.

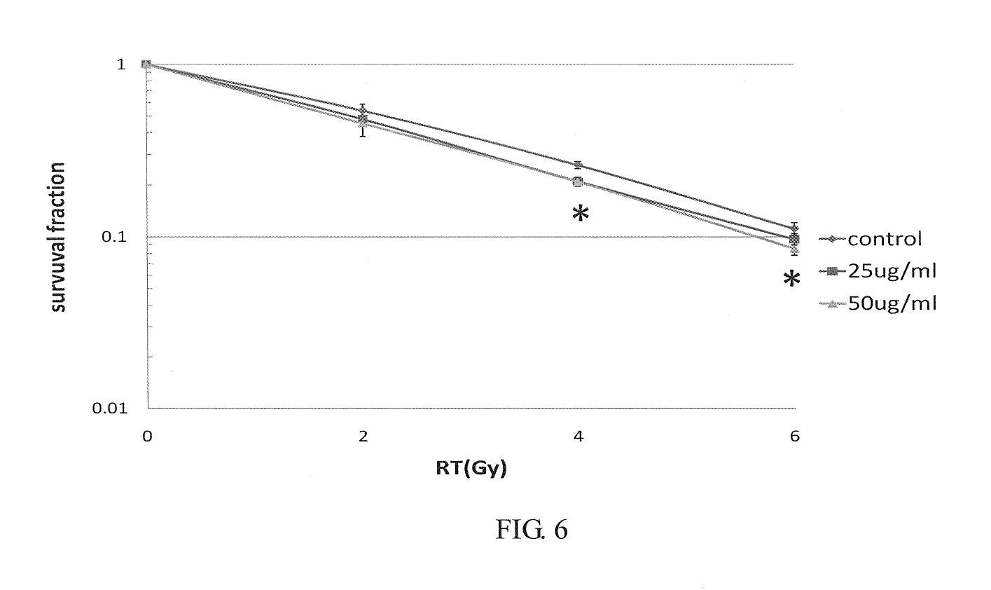

In combination of a radiation of 2, 4 and 6 Gy, respectively, U87 MG treated with 25, or 50 μg/ml ES800 exhibited significantly lower survival fraction, as compared with the control group treated with a radiation alone, see FIG. 6. It was evidenced that ES800 in combination with a radiation therapy provided good effectiveness for cancer treatment of any kind of a tumor.

### Example 7

### In Vitro Study of Schisandrin B as a Radiosensitizer

HepG2 cells were seeded in 6-well plate (2.5×10⁵ cells/well) for 24 hours of incubation. Various concentrations of ES800 (40 or 80 μg/ml) and Schisandrin B (12 or 24 μg/ml) were added into the culture medium, and then incubated for another 2 hours, wherein control group was added 0.008% DMSO. The cells were exposed at 8 Gy of radiation (Linear accelerator, Philips SL-18), and incubated for another 70 hours. After collection, the cells were washed by pre-cold PBS and centrifuged at 500×g. Following removing the supernatant, the cells were suspended with binding buffer. 1 μl Annexin V-FITC solution and 5 μl PI solution were added into the suspension and the mixture was reacted away from light on ice for 15 min. Finally, 400 μl pre-cold binding buffer was added and the samples were analyzed by flow cytometer within 30 min. The percentages of Annexin V⁺ and PI^{+/-} double staining cells were plotted in FIG. 7.

As shown in FIG. 7, Schisandrin B (12 or 24 μg/ml) without an exposure of radiation showed no effect on the apoptosis of cancer cells. However, the apoptosis ratio of HepG2 treated with Schisandrin B (12 or 24 μg/ml) in combination with a radiation was significantly higher than that of the control group. Furthermore, the apoptosis ratio of HepG2 treated with 24 μg/ml of Schisandrin B in combination with a radiation was significantly higher than that of HepG2 treated with a radiation of 8 Gy alone. It was indicated that Schisandrin B is also a potential radiosensitizer.

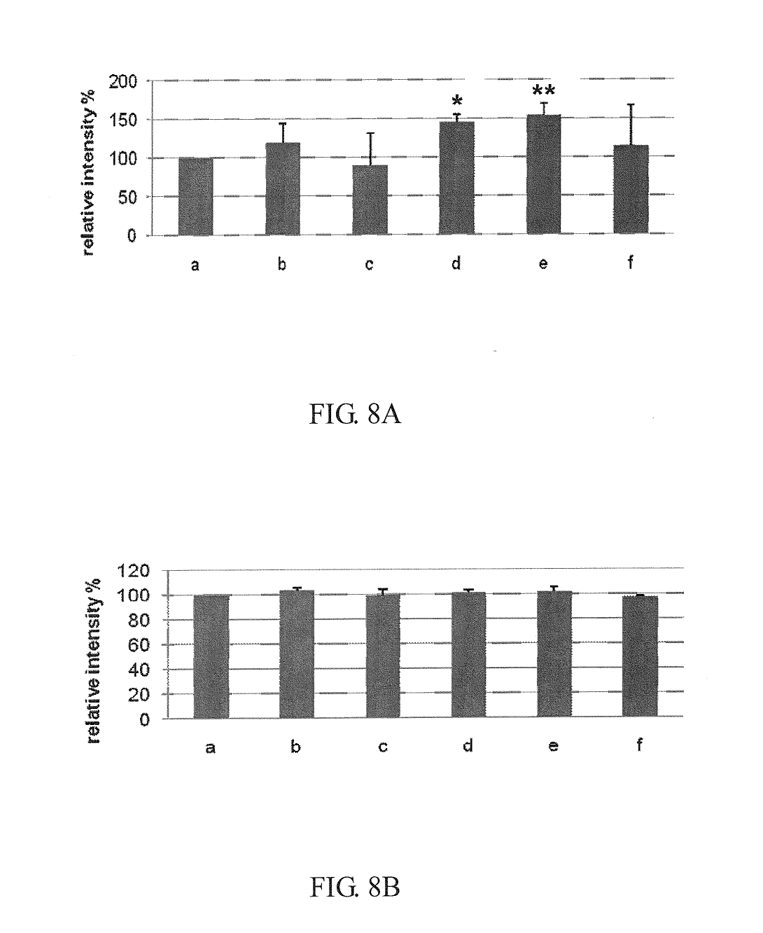

The expression level of apoptosis protein in HepG2 was also detected by western blot. The protocol of performing western blot is the same as described in Example 4. As shown in FIG. 8, the expression level of caspase 3 in HepG2 cells treated with 12 μg/ml Schisandrin B in combination with a radiation was significantly higher than that of the control group treated with a radiation alone, which showed the effect of Schisandrin B in potentiating radiation therapy in inducing apoptosis of cancer cells. β-actin was served as an internal control and each signal was normalizing to that of β-actin.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of potentiating radiation therapy for treatment of a cancer or tumor comprising administrating to a subject in need thereof a therapeutically effective amount of a radiosensitizer in combination of a radiation therapy to a locus of the cancer or tumor, wherein the radiosensitizer is an extract of Schisandra chinensis (Turcz.) Baill.

2. The method of claim 1, wherein the extract of Schisandra chinensis (Turcz.) Baill is prepared by a process comprising the steps of:
(a) extracting Schisandra chinensis (Turcz.) Baill with water to obtain a water insoluble fraction;
(b) extracting the water insoluble fraction obtained in step (a) with alcohol-based solvent to obtain an alcohol extract;
(c) removing the alcohol-based solvent from the alcohol extract obtained in step (b).

3. The method of claim 2, wherein the alcohol-based solvent in step (b) is ethanol.

4. The method of claim 1, wherein the cancer is a solid cancer.

5. The method of claim 1, wherein the cancer is liver cancer or brain cancer.

6. A method of potentiating radiation therapy for treatment of a cancer or tumor comprising administrating to a subject in need thereof a therapeutically effective amount of a radiosensitizer in combination of a radiation therapy to a locus of the cancer or tumor, wherein the radiosensitizer is a compound of formula (I): 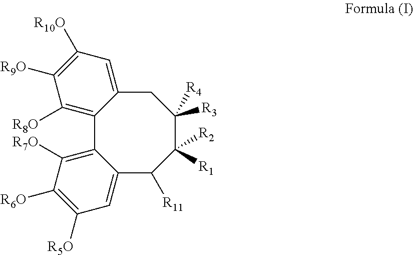
wherein one of R₁ to R₁₀ is H or C₁-C₃ alkyl, and R₁₁ is —OH, —O-benzoyl, —O-angeloyl, or —O-tigloyl, wherein R₅ and R₆ or R₉ and R₁₀ may be taken together with the adjacent oxygen and the carbon to which the oxygen atoms are bound to form 1,3, dioxole.

7. The method of claim 6, wherein the compound is selected from the group consisting of Gomisin O, Epi-gomisin O, Schisandrin, Isoschisandrin, Schizandrol B, Gomisin R, Gomisin J, Gomisin G, Schisantherin A, Gomisin F, Angeloylgomisin P, Tigloylgomisin P, Schisanhenol, Deoxyschisandrin, Gomisin N, Schisandrin B, Gomisin M1, Gomisin M2, Gomisin L1, Gomisin L2, and Schisandrol A

8. The method of claim 7, wherein the compound is Schisandrin B.

9. The method of claim 6, wherein the cancer is a solid cancer.

10. The method of claim 6, wherein the cancer is the cancer is liver cancer or brain cancer.
